(19) United States
(12) Patent Application Publication
(10) Pub. No.: US 2006/0183986 A1
(43) Pub. Date: Aug. 17, 2006

(54) Intraocular lens measurement of blood glucose

(76) Inventors: Mark J. Rice, Jacksonville, FL (US); John L. Smith, Fair Play, CA (US)

Correspondence Address: WILSON SONSINI GOODRICH & ROSATI, 650 PAGE MILL ROAD, PALO ALTO, CA, 94304-1050, US

(21) Appl. No.: 11/057,080

(22) Filed: Feb. 11, 2005

(51) Int. Cl. A61B 5/00

(52) U.S. Cl. 600/319

(57) ABSTRACT

One aspect of the invention provides a blood glucose monitoring system including a light source adapted to transmit light onto at least a portion of a retina of an eye of a subject; a sensor adapted to receive light from the retina; a data capture and analysis system adapted to calculate blood glucose concentration of the subject from the light received by the sensor; and support structure adapted to maintain positions of the light source and the sensor; wherein at least one of the light source and the sensor is further adapted to be implanted within the eye. Another aspect of the invention provides a method of determining blood glucose concentration of a subject including the steps of: transmitting light to a retina of an eye of the subject; receiving reflected light from the retina; and calculating blood glucose concentration from the reflected light, wherein one or both of the light source and sensor are disposed within the eye. The invention also provides a method of implanting a blood glucose monitor as well as a blood glucose monitor adapted to receive information from a sensor disposed within a subject's eye.

### BACKGROUND OF THE INVENTION

This invention pertains to the field of measurement of blood analytes such as glucose. The measurement of blood glucose by patients with diabetes has traditionally required the drawing of a blood sample for in vitro analysis. The blood sampling is usually done by the patient himself as a finger puncture, or in the case of a young child, by an adult. The need to draw blood for analysis is undesirable for a number of reasons, including discomfort to the patient, the high cost of glucose testing supplies, and the risk of infection with repeated skin punctures which results in many patients not testing their blood as frequently as recommended.

Many of the estimated three million Type I diabetics in the United States are asked to test their blood glucose up to six times or more per day in order to adjust their insulin doses for tighter control of their blood glucose levels. As a result of the discomfort, many of these patients do not test as often as is recommended by their physician, with the consequence of poor blood glucose control. This poor control has been shown to result in increased complications from this disease. Among these complications are blindness, heart disease, kidney disease, ischemic limb disease, and stroke. In addition, there is recent evidence that Type II diabetics (numbering over 12 million in the United States) may reduce the incidence of diabetes-related complications by more tightly controlling their blood glucose. Accordingly, these patients may be asked to test their blood glucose nearly as often as the Type I diabetic patients.

It would thus be desirable to obtain fast and reliable measurements of blood glucose concentration through more simple testing, without the need for repeated blood drawing. Prior efforts to obtain non-invasive blood glucose measurements have typically involved the passage of light waves through solid tissues such as the fingertip, forearm and the ear lobe and subsequent measurement of the absorption spectra. These efforts have been largely unsuccessful primarily due to the variability of absorption and scatter of the light waves in the tissues. These approaches, which have generally attempted to measure glucose concentration by detecting extremely small optical signals corresponding to the absorbance spectrum of glucose in the infrared or near-infrared portion of the electromagnetic spectrum, have suffered from the size requirements of instrumentation necessary to separate the wavelengths of light for this spectral analysis. Some groups, as illustrated by U.S. Pat. No. 6,280,381, have reported the use of diffractive optical systems, while others, as illustrated by U.S. Pat. No. 6,278,889, have used Fourier-transform or interferometric instruments. Regardless of approach, the physical size and weight of the instruments described have made it impractical for such a device to be hand-held or worn on the body as a pair of glasses. Other groups have attempted non-invasive blood glucose measurement in body fluids such as the anterior chamber of the eye, tears, and saliva. More recent developments have involved the analysis of light reflected from the retina of the eye to determine concentrations of blood analytes. See U.S. Pat. Nos. 6,305,804; 6,477,394; and 6,650,915.

A glucose measurement that could be made without drawing blood would be very advantageous for several reasons including the elimination of the constant pain and hassle from blood drawing and the ongoing cost of the glucose measurement strips required with blood testing. U.S. Pat. No. 6,650,915, US 2004/0147820A1, and US 2004/0087843A1 describe the noninvasive measurement of blood glucose through a novel method using the retina. In these descriptions, the retina is illuminated with an external device from outside of the eye and light is reflected from the retina back through the pupil, where it is collected, analyzed, and used to calculate the subject's blood glucose concentration.

The placement of an intraocular lens (IOL) for treatment of cataract has become a low-risk as well as very common procedure. During this surgical procedure, the native lens is extracted and an artificial lens (the IOL) is placed into either the same position as the native lens or into the anterior chamber of the eye. By way of reference, U.S. Pat. No. 2,834,023 describes an IOL placed into the anterior chamber of the eye. U.S. Pat. No. 3,866,249 describes an IOL placed-into the posterior chamber of the eye.

Increasingly, devices which compress flexible or “foldable” intraocular lenses and insert them into the eye through very small (3.5 mm) incisions are being employed by surgeons, as described in U.S. Pat. No. 6,251,114 to Farmer, et al. Patients with diabetes have a much higher incidence of cataracts than the general population and subsequently, undergo IOL surgery more commonly. In addition, IOLs are now emerging in the marketplace that can change in shape or position, allowing patients with presbyopia (the loss with age of the ability to accommodate for close vision) the opportunity to recover this ability and discard their reading glasses. An example is described in U.S. Pat. No. 6,387,126 to Cumming. Since the advent of these accommodating IOLs, patients without cataracts are beginning to have IOL replacements for vision correction as well as for the treatment of cataracts.

A further development in the field of intraocular lenses has been the use of lenses in addition to, rather than in place, of the natural lens in the eye in order to provide vision correction for people with vision problems too extreme to be corrected by the use of eyeglasses or contact lenses. By providing additional magnification, normal vision can be restored when such an intraocular contact lens is inserted into the posterior chamber of the eye, in front of the natural lens. An example of this approach is described in U.S. Pat. No. 6,106,553 to Feingold.

March U.S. Pat. No. 6,681,127 describes the use of an ophthalmic lens (contact lens or IOL) to measure glucose by binding this analyte with a receptor moiety bound to the surface of the lens to determine the amount of glucose in a fluid related to the eye. Frenkel U.S. Pat. No. 5,005,577 was one of the first to describe an intraocular lens which contained a pressure transducer combined with a transponder system for noninvasive measurement of intraocular pressure. Other patent documents have subsequently published which are directed to similar devices for measurement of intraocular pressure. Abreu U.S. 2004/0039298A1 describes the use of intraocular lenses to measure glucose in the aqueous humor or other fluid of the eye, but does not disclose measuring light reflected from the retina or the use of visual pigments as a means of determining glucose concentration.

### SUMMARY OF THE INVENTION

The subject matter of this invention pertains to the measurement of blood glucose using the rate of depletion or regeneration of visual pigments measured by photometric means by placing a light source and/or detector into the body of an IOL. One aspect of the invention provides a blood glucose monitoring system including a light source adapted to transmit light (e.g., light in wavelengths absorbed by visual pigment) onto at least a portion of a retina of an eye of a subject, such as the fovea; a sensor adapted to receive light from the retina; a data capture and analysis system adapted to calculate blood glucose concentration of the subject from the light received by the sensor; and support structure adapted to maintain positions of the light source and the sensor; wherein at least one of the light source and the sensor is further adapted to be implanted within the eye.

In embodiments in which the sensor is adapted to be implanted within the eye, the system may further include a lens adapted to direct light from the retina onto the sensor. In some embodiments, the sensor may have a substantially toroidal shape. In other embodiments, the sensor is adapted to transmit data outside of the eye to the data capture and analysis system. In some embodiments, the light source and the sensor are both adapted to be implanted within the eye.

In some embodiments, the data capture and analysis system is adapted to be disposed outside of the eye. The data capture and analysis system may also include a display adapted to display glucose concentration information. The data capture and analysis system may also include a receiver adapted to receive information from the sensor and/or a transmitter adapted to transmit power to the light source. In some such embodiments, the receiver may be further adapted to receive unique identification information from the sensor. In other such embodiments, the transmitter may be further adapted to transmit unique identification information to the sensor.

In some embodiments, the support structure includes light source support structure adapted to support the light source behind an iris of the eye and in front of a natural lens of the eye. In other embodiments, the support structure includes light source support structure adapted to support the light source behind an iris of the eye and in place of a removed natural lens of the eye. In some embodiments, the support structure includes light source support structure, with the system further including an implant tool adapted to insert the light source and light source support structure into the eye. In still other embodiments in which the light source is adapted to be disposed outside the eye and the sensor is adapted to be implanted within the eye, the support structure includes light source support structure adapted to support the light source in front of the eye and sensor support structure adapted to support the sensor within the eye.

In some embodiments, the system also includes an insulin source (such as a pump) adapted to provide insulin to the subject in response to blood glucose concentration calculated by the data capture and analysis system. Such systems may also include an alarm adapted to provide an indication of an upcoming transmission from the light source and an optional light transmission override adapted to be operated to temporarily prevent transmission from the light source to the subject. Some embodiments of the system of this invention may also have an alarm adapted to provide notice to the subject as a result of a blood glucose concentration calculated by the data capture and analysis system.

Another aspect of the invention provides a method of determining blood glucose concentration of a subject including the steps of: transmitting light to a retina of an eye of the subject from a light source within the eye; receiving reflected light from the retina; and calculating blood glucose concentration from the reflected light. The method's transmitting step may include the step of directing light on the retina, such as on a foveal portion of the retina.

In some embodiments, the receiving step includes the step of receiving light reflected from the retina (in some embodiments, from a foveal portion of the retina) in a sensor disposed within the eye. In other embodiments, the receiving step includes the step of receiving light reflected from the retina in a sensor disposed outside the eye. In some embodiments, the method may include the step of activating an alarm based on a result of the calculating step.

In some embodiments, the calculating step includes the step of determining a rate of change of light reflected from the retina. In embodiments in which the receiving step is performed by a sensor and the calculating step is performed at least in part by a data capture and analysis system, the method may further include the step of transmitting information related to light reflected from the retina from the sensor to the data capture and analysis system. In embodiments in which the sensor is disposed within the eye and the data capture and analysis system is disposed outside of the eye, the step of transmitting information may include the step of transmitting the information from within the eye to outside the eye. In some embodiments, the step of transmitting information may include the step of transmitting the information from within the eye to outside the eye through a closed eye. In still other embodiments, the step of transmitting information includes the step of transmitting unique identification information from the sensor to the data capture and analysis system. In other embodiments, the method includes the step of transmitting unique identification information from the data capture and analysis system to the sensor.

Some embodiments of the method include the step of transmitting power from a source external to the eye to the light source. Some embodiments include the step of preventing ambient light from entering the eye during the receiving step. Still other embodiments include the step of displaying blood glucose concentration.

Some embodiments of the method include the step of automatically controlling administration of insulin from an insulin source to the subject based on blood glucose concentration. In some embodiments, the method includes, prior to the transmitting step, the step of providing notice to the subject of an upcoming transmitting step and possibly the step of delaying the performance of the transmitting step.

Yet another aspect of the invention provides a method of determining blood glucose concentration of a subject including the following steps: transmitting light to a retina of an eye of the subject; receiving reflected light from the retina in a sensor disposed within the eye; and calculating blood glucose concentration from the reflected light. The method may also include the step of activating an alarm based on a result of the calculating step.

In some embodiments, the transmitting step includes the step of directing light on a foveal portion of the retina. In some embodiments, the receiving step may include the step of receiving light reflected from a foveal portion of the retina. In some embodiments, the transmitting step includes the step of transmitting light to the retina from a light source disposed outside the eye.

In some embodiments, the calculating step includes the step of determining a rate of change of light reflected from the retina. In still other embodiments, the calculating step is performed at least in part by a data capture and analysis system external to the eye, with the method further including the step of transmitting information related to light reflected from the retina from the sensor to the data capture and analysis system, such as through a closed eye.

In some embodiments, the step of transmitting information may include the step of transmitting unique identification information from the sensor to the data capture and analysis system and/or from the data capture and analysis system to the sensor. The method may also include the step of displaying blood glucose concentration.

In some embodiments, the method includes the step of automatically controlling administration of insulin from an insulin source to the subject based on blood glucose concentration. The method may also include the step of, prior to the transmitting step, providing notice to the subject of an upcoming transmitting step and optionally delaying the performance of the transmitting step.

Yet another aspect of the invention provides a method of implanting a blood glucose monitor including the steps of: providing an implantable blood glucose monitor comprising a light source; inserting the implantable blood glucose monitor through an outside surface of an eye (such as into an anterior chamber or a posterior chamber of the eye); and orienting the light source in a position from which the light source can illuminate at least a portion of a retina of the eye. In some embodiments, the providing step includes the step of providing an implantable blood glucose monitor having a light source and a sensor adapted to receive light, with the orienting step further including the step of orienting the sensor to receive light from the retina.

In other embodiments of the invention, the inserting step includes the step of supporting the implantable blood glucose monitor with an insertion tool. In such embodiments, the supporting step may also include the step of supporting the implantable blood glucose monitor in an insertion state, with the method further including the step of moving the implantable blood glucose monitor from the insertion state to a deployed state.

Still another aspect of the invention provides a blood glucose monitor including: a receiver adapted to be disposed outside of an eye and to receive information related to light reflected from a retina of the eye from a sensor disposed within the eye; and a processor adapted to calculate blood glucose concentration from the information. In some embodiments, the receiver may be further adapted to receive through a closed eye information related to light reflected from the sensor. Some embodiments of this aspect of the invention may include a display adapted to display blood glucose concentration calculated by the processor. Still other embodiments may include a transmitter adapted to transmit power to a light source implanted in the eye. In some embodiments, the blood glucose monitor also includes a transmitter adapted to transmit a signal related to calculated blood glucose concentration to an insulin source.

Another aspect of the invention provides a blood glucose monitor including: a sensor adapted to be disposed outside of an eye and to receive light reflected from a retina of the eye from a light source disposed within the eye; and a processor adapted to calculate blood glucose concentration from the information. In some embodiments, the blood glucose monitor further includes a display adapted to display blood glucose concentration calculated by the processor. In some embodiments, the blood glucose monitor includes a transmitter adapted to transmit power to a light source implanted in the eye.

Further objects, features, and advantages of the invention will be apparent from the following detailed description when taken in conjunction with the accompanying drawings.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a front view diagram of the eye.

FIG. 2 is a top view diagram of the eye.

FIG. 3 is a front-view diagram of an intraocular lens apparatus for measurement of blood glucose in accordance with an exemplary embodiment.

FIG. 4 is a side-view diagram of an intraocular lens apparatus for measurement of blood glucose in accordance with an exemplary embodiment.

FIG. 5 is a top view diagram of the eye and intraocular lens showing an exemplary embodiment of a system for measurement of blood glucose.

FIG. 6 is a front view of the eye with the IOL in place.

FIG. 7 is a diagram of the patient holding a fob near his head for glucose measurement with the invention.

FIG. 8 is a diagram of the eye and IOL showing the light path from the light source to the retina and then, following reflection from the retina, back to the sensor contained in the IOL.

FIG. 9 is a diagram of an alternative embodiment of the IOL showing a larger sensor in the shape of a doughnut.

FIG. 10 is a diagram showing elements of an alternative embodiment of the invention.

FIG. 11 is a diagram of a curve showing the trace of reflectance that would result from the bleaching of visual pigment by bright light, followed by the measurement of the regeneration of visual pigment using less intense light.

FIG. 12 is a diagram of one possible sequence of foveal illumination during the measurement.

FIG. 13 is a diagram of a folded IOL, held by a surgical instrument, just prior to implantation.

FIG. 14 is a diagram of a system used for insertion of intraocular lenses into the posterior chamber of the eye.

FIG. 15 is a diagram of an intraocular lens which provides accommodation and containing the optical elements of a glucose measurement system.

FIG. 16 is a diagram of an intraocular contact lens used to provide vision for people with severe vision problems, containing the elements of a glucose measurement system.

FIG. 17 is a schematic representation of a closed loop system according to one embodiment of this invention.

### DETAILED DESCRIPTION OF THE INVENTION

Rhodopsin is the visual pigment contained in the rods that allows for dim vision. Cone visual pigments (sometimes termed “opsins”) are contained in the cones of the retina and allow for central and color vision. The outer segments of the rods and cones contain large amounts of visual pigment, stacked in layers lying perpendicular to the light incoming through the pupil. As visual pigment absorbs light, it breaks down (bleaches) into intermediate colorless molecular forms and initiates a signal that proceeds down a tract of nerve tissue to the brain, allowing for the sensation of sight. This phenomenon is termed bleaching, since the retinal tissue loses its color content when a light is directed onto it.

The colorless compounds formed during the bleaching process (which is a consequence of vision and therefore a continuous process whenever light enters the eye) are converted back to the original, colored compounds in a sequence of chemical reactions called “regeneration.” Like the bleaching of pigments that accompanies vision, the regeneration process also occurs continuously, even during bleaching. Rod visual pigment absorbs light energy in a broad band centered at 500 nm, whereas the three different cone visual pigments have broad overlapping absorption bands peaking at 430, 550, and 585 nm, which correspond to blue, green, and red cones, respectively. (These cones are also known as the short wavelength, medium wavelength, and long wavelength cones, respectively.)

The rods and cones of the retina are arranged in specific locations in the back of the eye. The cones, which provide central and color vision, are located with their greatest density in an area of the retina called the fovea, and especially in a central feature of the fovea termed the fovea centralis or foveola. The fovea covers a circular area with a diameter of about 1.5 mm, and the fovea centralis covers an area with a diameter of approximately 0.6 mm. The rods are found predominately in the more peripheral portions of the retina and contribute to vision in dim light. For purposes of this patent application, “fovea” includes “fovea centralis” and “foveola.”

Visual pigment consists of 11-cis-retinal and a carrier protein, which is tightly bound in either the outer segment of the cones or rods. 11-cis-retinal is the photoreactive portion of visual pigment, which is converted to all-trans-retinal when a photon of light in the active absorption band strikes the molecule. During the regeneration process, all-trans-retinal is isomerized back to 11-cis-retinal as detailed below.

Following the photoconversion of 11-cis-retinal to all-trans-retinal, the 11-cis-retinal is regenerated by a series of steps that result in 11-cis-retinal being recombined with an opsin protein. A critical (and rate-limiting) step in this regeneration pathway is the reduction of all-trans-retinal to all-trans-retinol catalyzed by the enzyme all-trans-retinol dehydrogenase (ATRD), which requires NADPH as the direct reduction energy source. In a series of experiments, Futterman et al. have proven that glucose, via the pentose phosphate shunt (PPS), provides virtually all of the NADPH needed for this critical reaction. S. Futterman, et al., “Metabolism of Glucose and Reduction of Retinaldehyde Retinal Receptors,” J. Neurochemistry, 1970, 17, pp. 149-156. Without glucose or its immediate metabolites, only very small amounts of NADPH are formed and visual pigment cannot regenerate.

In addition, Ostroy, et al. have proven that visual pigment regeneration is strongly dependent on the extracellular glucose concentration. S. E. Ostroy, et al., “Extracellular Glucose Dependence of Rhodopsin Regeneration in the Excised Mouse Eye,” Exp. Eye Research, 1992, 55, pp. 419-423. When glucose is plentiful (at high blood glucose), the rate of regeneration of visual pigment is very high, conversely at low blood glucose levels, the rate of regeneration is reduced. Embodiments of the present invention utilize this relationship to measure blood glucose concentrations. A number of specific measurement methodologies have been described in pending U.S. patent application Ser. No. 10/863,619 to make an accurate measurement of the visual pigment regeneration rate. More than one method may be chosen for use with embodiments disclosed herein.

The present invention carries out measurements of blood glucose in a repeatable manner by measurement of the rate of depletion or regeneration of retinal visual pigments, such as cone visual pigments. As stated above, the rate of regeneration of visual pigments is dependent upon the blood glucose concentration, by virtue of the glucose concentration limiting the rate of production of a cofactor, NADPH, which is utilized in the rate-determining step of the regeneration of visual pigments. Thus, by measuring the visual pigment regeneration rate, blood glucose can be accurately determined. One embodiment of this invention exposes the retina to light of selected wavelengths at selected times and analyzes the reflectance from a selected portion of the retina, preferably from the fovea.

In one embodiment, a light source (such as a light emitting diode or LED) and a detector (such as an array photodetector) are housed in an IOL, which is placed in the posterior chamber of the eye. The surgery for placement of the IOL may be performed, e.g., as part of treatment of cataract or presbyopia or may be performed solely to implant the blood glucose monitoring device of this invention. The light source and the detector, which may be a PIN (Positive-Intrinsic-Negative) diode, APD (Avalanche Photodiode), CCD (Charge-Coupled Device array), CMOS (Complementary Metal-Oxide Semiconductor array) or other sensor, are placed toward the periphery of the IOL, facing the retina, such that they are masked by the edge of the iris and thus not visible to the subject. The device also includes an induction coil (functioning as a receiving antenna) wound into the periphery of the IOL, again so as not to be visible to and cause distraction for the subject. This induction coil obtains power through magnetic induction or radio frequency energy from an externally-generated source to power the light source, sensor and other electronics contained in the IOL. A small device such as a key fob may be used by the subject to generate the magnetic or radio frequency field that supplies power, to initiate the measurement sequence, to receive information from the IOL and to calculate the subject's blood glucose concentration. The fob contains a power source, display, transceiver and logic for interaction with the components contained in the IOL. The subject holds the small fob near the forehead and initiates the series of events required for glucose measurement as described below. In some embodiments, the fob has an alphanumeric display for displaying the results of the glucose measurement.

In some embodiments, the system's power source, transceiver, and logic is also connected to a device such as a pump that supplies appropriate amounts of insulin as a self-contained “closed loop” system to function as an artificial pancreas.

The light source that is used to generate the illuminating light may be directed onto a portion of the retina by a lenslet (a small, secondary lens molded into or attached to the posterior surface of the IOL) that illuminates the fovea for subsequent analysis. Alternatively, the non-foveal retina may be used to measure pigment regeneration.

In some embodiments of the invention, a photodetector or photodetector array such as a PIN diode, APD, CCD or CMOS is used to measure the light returned from the region of the fovea (through a second lenslet on the IOL) to determine the rate of depletion or regeneration of retinal pigments such as the cone visual pigments. In another embodiment where the non-foveal region is illuminated, the light reflected from that region is directed to the detector and analyzed to determine glucose concentration.

With this invention, light from the source may be used to break down (deplete or bleach) the visual pigment, and light reflected from the retina can be subsequently analyzed over a period of time to determine the regeneration rate of the visual pigment. Some embodiments of this invention use a light source that varies in a selected temporal manner, such as a periodically applied stimulus of light that may break down the visual pigment. Light reflected from the retina is analyzed over a period of time to determine the regeneration rate of the visual pigment. As the pigment is depleted during bleaching, the color or darkness of the retina decreases (that is, the retina becomes lighter in color), with the result that more light is reflected by the bleached retina (increased reflectance). During regeneration, the pigment is restored over time, making the retina progressively darker and less reflective of light, producing decreases in reflectance as the regeneration proceeds.

When modulated light is used in conjunction with synchronous (frequency-selective) detection, the measurement is made without excluding light from the eye. If a detector is employed with enough dynamic range to sense the variations due to modulation in addition to the ambient light, the ambient light does not significantly affect the measurement, since it will have little or no content within the frequency band of the modulation. Thus, a patient's eye can be open during a glucose measurement in reasonable light levels.

Measurement of an unknown blood glucose concentration is accomplished by development of a relationship between the reflected light data (indicating the visual pigment regeneration rate) and corresponding clinically determined blood glucose concentration values. With one embodiment of this invention, a steady-state illuminating light or a varying illuminating light may be applied to induce bleaching and a steady-state illuminating light or a varying illuminating light may be applied to determine the regeneration rate of the visual pigment. Measurement of the regeneration rate may also be accomplished during the bleaching phase, as regeneration of the visual pigments occurs even while the pigments are being bleached. In addition, measurement of visual pigment regeneration may be made without a formal bleaching event. Pulsed or other light-varying techniques may be used to measure the regeneration rate of the visual pigment. Light modulated in a number of ways, such as by sinusoidal, square wave or pulsed techniques, may also be used to observe the processes.

An additional advantage of the IOL approach is that the patient is not required to look toward a source of light or in any particular direction, since alignment between the IOL and the retina is maintained at all times independent of the direction of gaze of the patient.

There are several modes of measuring glucose with a source and detector inside the eye that are not possible when the measurement is made using light introduced from outside the eye. Unlike an external measurement, a device based on an IOL may be used to make “continuous” measurements of glucose. “Continuous” measurements are typically measurements taken repeatedly at relatively short intervals. When a signal is provided to the user that it is time to make a measurement, the only action required by the user is to close the eye or otherwise exclude light for the period of time required to make a measurement. When the measurement is made using modulated light, even this action would be unnecessary. If making a measurement at a particular time is inconvenient for the user (during driving, conversation or other absorbing activity), he would indicate by pressing a button, or simply continuing to allow ambient light to enter the eye. This indication would be detected by the detection system, allowing the measurement to be postponed to a later time. Another alternative is for the patient not to bring an external power source close enough to the eye to initiate a measurement sequence.

Measurements can also be made at night by using light at either the blue or red end of the spectrum where visual perception is less sensitive. Once a patient becomes adapted to the periodic light falling on the eye, it is possible that sleep would not be disturbed and measurements could be made throughout the night while the patient is asleep. These measurements may be used to provide glucose information to control the output of an insulin pump, providing a closed-loop system that functions as an artificial pancreas. Alternatively, during waking hours, the information could be displayed or otherwise provided to the patient to guide dosage of insulin or other diabetes medication.

Unlike external illumination, which must pass through the cornea, crystalline lens, and other interfaces in the eye, illumination from an IOL could be of any wavelength, provided that the intensity and duration were within established safety limits. This would allow light in the blue region to be used, where it would be precluded in external illumination because blue light is scattered to a much greater degree than light of higher wavelengths, especially by cataracts. In addition, the alignment between the optical system of the devices described here and the retina is maintained at all times by the fixed position of the IOL relative to the lens. As a result, head or eye motion does not alter the optical alignment and does not interfere with making a measurement.

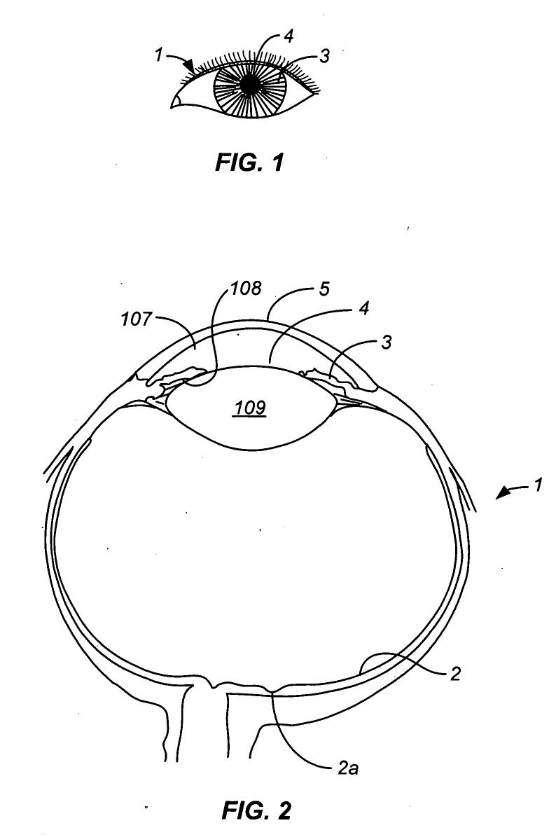

With reference to the drawings, FIG. 1 illustrates the eye 1 from a front view. The iris 3 is a circular colored structure that surrounds the pupil 4. The diameter of the pupil is controlled by smooth muscle in the iris and varies with many different conditions, including ambient light level.

FIG. 2 is a cross-sectional top view of the eye 1. The front surface of the eye is called the cornea 5, which is a very thin membrane that allows light to enter the eye. Between the cornea and the iris is a fluid-filled space called the anterior chamber 107. The light then enters the pupil 4, which has a diameter determined by the size of the iris 3. Behind the iris is a fluid-filled space called the posterior chamber 108, and behind the posterior chamber is the natural (or crystalline) lens 109. Light passing through the iris is further focused by the lens, finally falling on the retina 2 at the back of the eye. The fovea 2a is the area of the retina that contains the highest concentration of cones and is the area of the highest visual acuity. It is located directly opposite the pupil 4. Both the anterior chamber and the posterior chamber are filled with a fluid, termed “aqueous humor.”

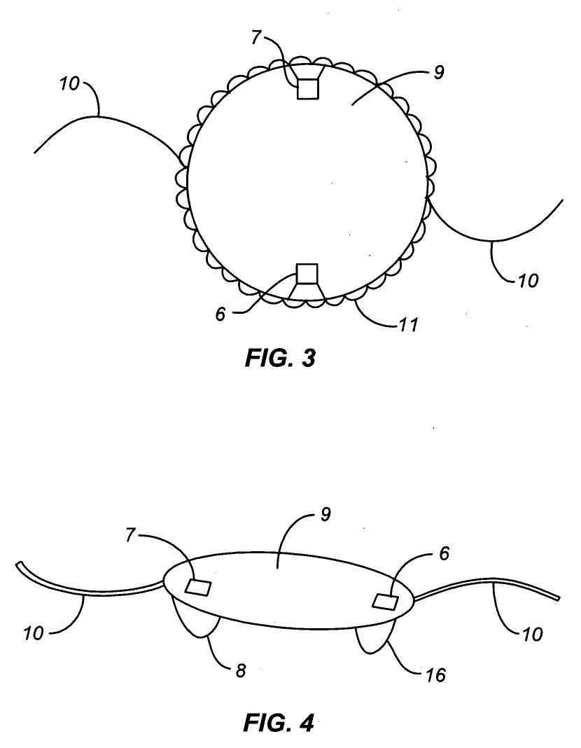

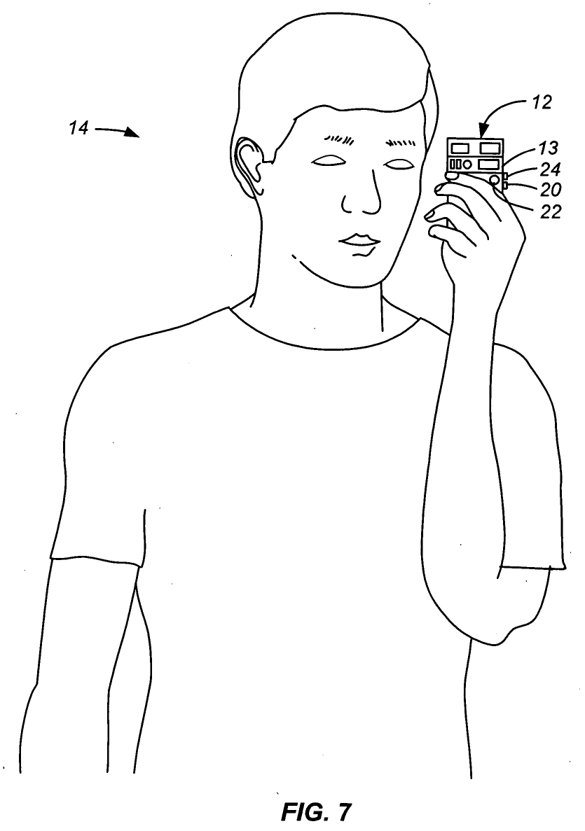

FIG. 3 shows an IOL 9 of one embodiment (front view) as it looks prior to surgical implantation into the eye. The IOL 9 contains an LED 7, a sensor (or multiple sensors) 6, and an induction coil 11 which serves both to obtain power through magnetic induction or radio frequency from an externally-generated source (such as fob 12 of FIG. 7) and as an antenna for receiving and transmitting data to the fob 12. The haptics 10 are flexible members extending from the body of the lens which are required for the IOL to remain fixed in the eye following surgical implantation. The haptics are known to those skilled in this art to be made in many different forms.

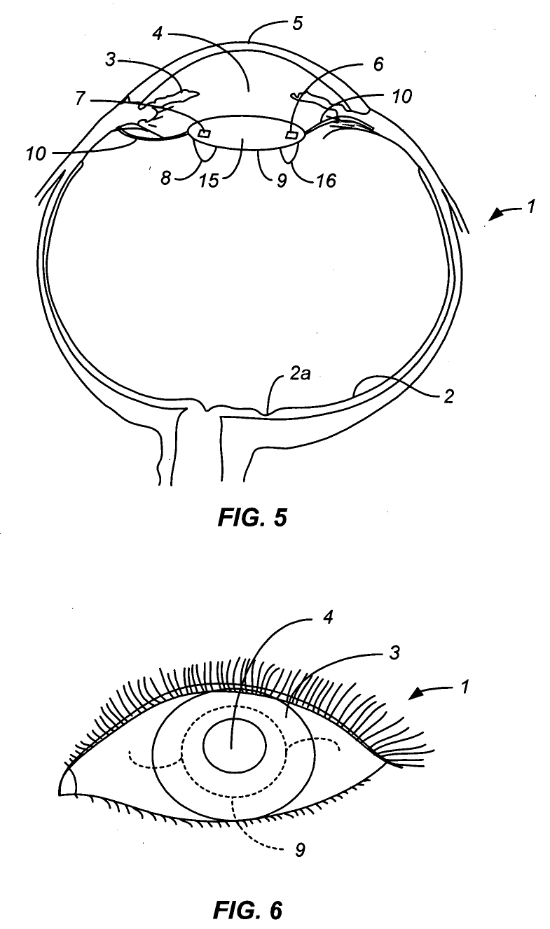

In FIG. 4, the side view of the IOL 9 is shown prior to surgical implantation. The LED 7 has an associated optional lenslet 8 that directs the light emitted from the LED 7 onto the fovea 2a (FIG. 5). An additional optional lenslet 16 directs light returning from the fovea 2a onto a sensor 6. The haptics 10 anchor the IOL into the eye following implantation.

FIG. 5 illustrates an embodiment of the present invention. The eye of the patient is illustrated at 1, with the optical system for directing light onto the retina and obtaining light reflected from the retina shown as IOL 9. The iris of the eye is shown as 3, with the pupil 4, and the fovea 2a. The IOL is made up of the optic 15 and the haptics 10, which keep the IOL in place following surgical placement. The illumination system is shown as LED 7 and contains the elements required for directing light from the light source (LED 7) preferably through an optional lenslet 8 on the posterior surface of the IOL and onto the retina for the breakdown of visual pigment (bleaching) and also for illumination during measurement of regeneration. The sensor is shown as element 6, which may be either a single element or multi-element detector. The data capture and analysis system comprises elements required for the measurement of the reflected light, calculation of the visual pigment regeneration rate, and conversion of this information into the blood glucose value; some of these functions are contained in a separate device, shown as a key fob 12.

In FIG. 6, the eye is illustrated from a frontal view after implantation of the IOL. As in FIG. 1, the eye 1 is shown with the iris 3 and the pupil 4. Behind the iris 3 and not visible from the outside is the IOL 9, which is diagrammed behind the iris as a dotted line on FIG. 6.

FIG. 7 shows a patient holding the fob 12 up to his temple area during the measurement process. In this embodiment, the fob contains the necessary source of power (which would be transferred to the IOL by magnetic induction or radio frequency), a radio frequency receiver to receive data from the IOL, logic, and provision for display of results via an LCD 13. The measurement sequence is initiated by the patient by pressing a button 20 on the fob or, alternatively, by merely bringing the fob into close proximity to the eye.

In some embodiments, the system takes measurements automatically on a preset schedule. In these embodiments, the system may provide notice to the subject of an impending measurement, such as by activation of an alarm (such as speaker 22 on fob 12). If a measurement would not be convenient at that time or would be in accurate because, for example, the subject could not close his or her eye during the measurement, the subject could delay that measurement event by depressing a pause or delay button 24 on fob 12.

In addition, in some embodiments the system also notifies the subject if the measured blood glucose concentration is outside of preset bounds. Such notice may be particularly important if the subject is not watching the displayed results of the blood glucose calculation, such as if the subject were asleep. Alarm 22 may be used for this purpose as well.

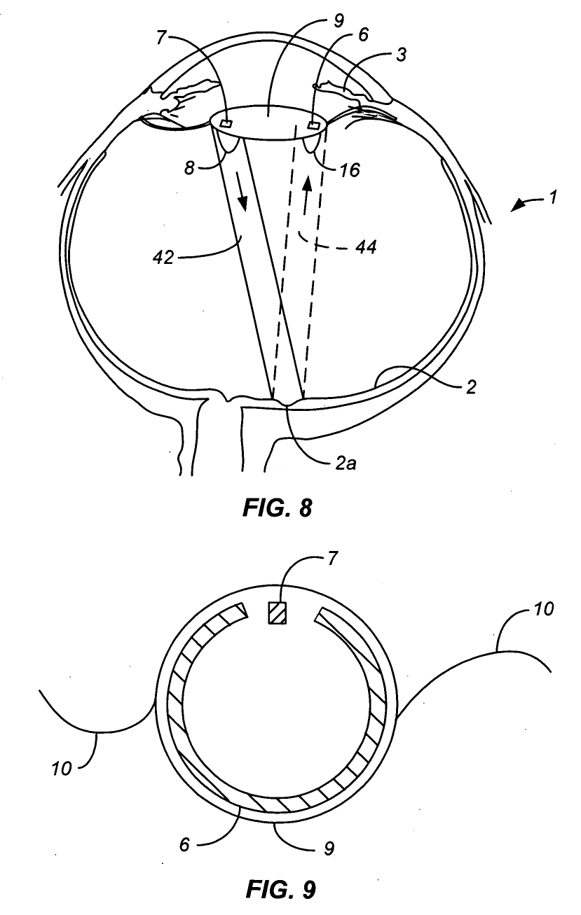

FIG. 8 illustrates the light path of the present invention. The illumination system (LED 7) provides selected illuminating light 42, through a lenslet 8, focused onto the retina, and preferably directed toward the fovea 2a. The LED 7 illuminates the selected portion of the retina with light that contains wavelengths which are absorbed by the visual pigments. The light from the illumination system is reflected from the retina (reflected light path 44) through the lenslet 16, onto a detector (sensor 6). The wavelength of this light source is selected dependent upon the particular visual pigment to be analyzed. Although generally any wavelength of light in the visible region of the spectrum (400-700 nm) could be used, the light intended for absorption by visual cone pigments could be centered at approximately 540 nm for green cones, 585 nm for red cones, and 430 nm for blue cones. Alternatively, light intended for absorption by rod rhodopsin could be centered at approximately 500 nm.

FIG. 9 illustrates another embodiment of the IOL. In an embodiment where light reflected from a large area of the retina is to be used for measurement, the sensor 6 could be “doughnut” shaped to increase the surface area and decrease the required light for detection. This would lessen the LED power requirements and increase the efficiency of the device. The sensor would have an internal diameter (ID) just larger than the patient's fully dilated (mydriatic) pupil diameter, so that in no case would the sensor be seen by the patient.

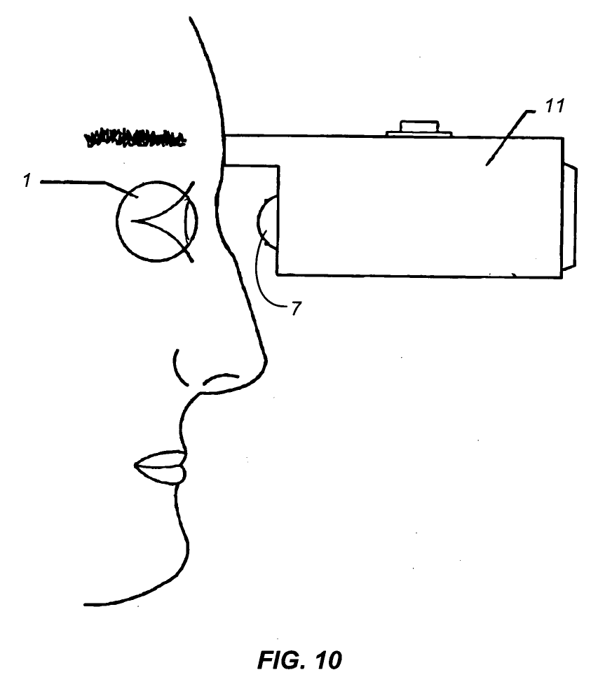

FIG. 10 illustrates an alternative embodiment in which the sensor is implanted and the light source is external. In this embodiment, the external fob 11 contains the light source 7 in addition to the data capture, analysis and display circuitry described with respect to embodiments above.

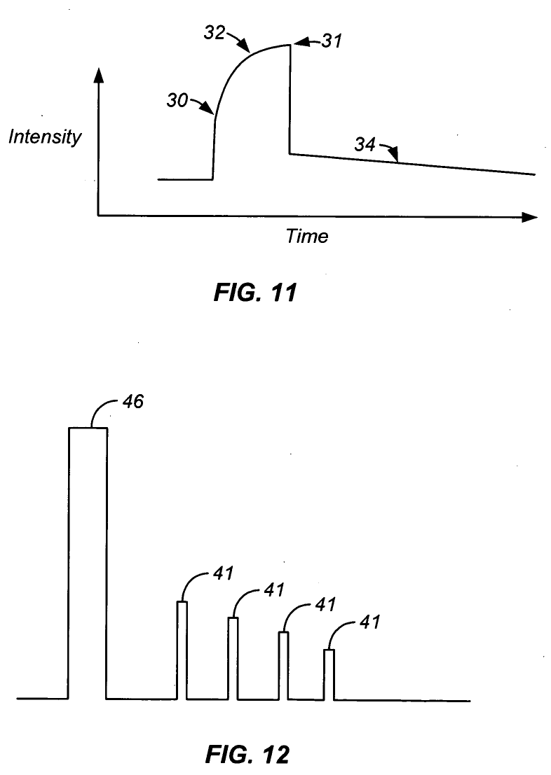

FIG. 11 illustrates an exemplary measurement sequence. The curve represents the amount of light reflected from a selected portion of the retina during the measurement sequence. The light is initially off or the light level is very dim, and at the time indicated by the rapid increase (30), the intensity of the light is increased to a level that will cause rapid bleaching of the visual pigments. The bleaching process is indicated by the increase in reflectance at 32. After a few seconds, the intensity of the light is reduced (31), and the reflectance of the retina is monitored during the regeneration process (34). As the pigment regenerates, the retina becomes darker and the amount of reflected light decreases, resulting in a downward slope of the curve. The rate of regeneration is dependent on the blood glucose concentration of the patient, with greater slopes corresponding to higher glucose levels. Even though there may be some noise or variation in the measured data (36), an accurate slope can be generated by preferably a simple linear regression of the data to yield a single straight line, 38. The slope of this line is used to calculate the blood glucose concentration for the patient.

To initiate a glucose measurement (see FIG. 7), the subject 14 holds the fob 12 near the temple and closes his eye. This keeps ambient light out of the eye, which could confuse the measurement. Alternatively, an eye patch or other occluding device could be placed in front of the eye to assist in eliminating ambient light. An initiation of the measurement is then made, in one embodiment, by pushing a button on the fob 12. Through an induction coil, power is transferred to the IOL from the fob and the illumination sequence is initiated. The sensor then collects the reflected light from the retina and transmits reflected light information to a receiver in the fob. Transmitters for transmitting information from an intraocular device to an external receiving device are known, as shown, e.g., in U.S. Pat. No. 6,443,893 and US 2004/0186366A1. In some embodiments, unique identification information (using, e.g., RFID (radio frequency identification) technology) is provided with the sensor transmission to identify the IOL to the fob. Alternatively, unique identification information may be transmitted from the fob's external receiver to the sensor in a similar fashion. The identification of sensor to fob or vice versa helps ensure accurate calculation of blood glucose concentration by making certain that the IOL and fob are intended to work together and by identifying the subject so that appropriate calibration information can be applied to the calculation. The resulting sensor data are collected by a radio frequency receiver in the fob and the blood glucose is calculated with logic embedded in the fob. The glucose result is displayed on the LCD screen 13. If the light measurement technique employed, such as modulated light with synchronous detection, is not adversely affected by reasonably steady ambient light, then the step of excluding ambient light could be omitted.

In some embodiments, the light transmission, reflected light sensing and blood glucose calculating is initiated by the system automatically. In these embodiments, the system may be provided with an alarm (such as speaker 22 in FIG. 7) to indicate an upcoming blood glucose measurement. The subject may override an upcoming light transmission and blood glucose measurement, such as by depressing an override button 24 in FIG. 7.

Following bleaching of the visual pigment with light at selected wavelengths, one embodiment uses the measurement of reflected light from the area of interest, which preferably is the fovea of the retina (although any area of the retina that contains visual pigment could be used) to measure visual pigment regeneration. The retina, at specific wavelengths of light, is illuminated as described above, and the reflected light is captured by a sensing device as described above. This sensing device may be a photodiode or any other device that can sense the amount of light being emitted from the eye (e.g., a CCD array) in order to measure the regeneration of the visual pigment during or following bleaching. In one embodiment, the light values of the pixels (in the case of a CCD) that are in a defined area containing the desired visual pigment to be measured can then be summed.

In some embodiments, the light source may be implanted in the eye within the IOL while the sensor is external to the eye, such as in the fob or other data capture and analysis system. In other embodiments, the sensor may be implanted in the eye within the IOL while the light source is external to the eye, such as described above with reference to FIG. 10.

Although the exemplary embodiments can be used to measure the changing light reflected off any defined area in the retina of the eye, it is preferred to measure the foveal area which contains the highest percentage of cones compared to rods. While both cones and rods contain visual pigment, the regeneration of cone pigment is considered to be faster than rod visual pigment regeneration and therefore preferable for measurement of regeneration rates. The highest concentration of cone visual pigment is contained in the area of the fovea.

Since several exemplary embodiments of this invention measure regeneration of visual pigment, the reflected light may be measured over a period of time, either with constant light or via a series of pulses. One embodiment makes the measurement of visual pigment regeneration with a series of pulses. This temporal measurement can be accomplished by comparing the reflected illumination from pulse to pulse, over a series of pulses, of the same area of the retina. A better estimate of the changing reflectance may be made by averaging the change in reflectance over a number of pulses to minimize noise. Although a large number of pulses may be used for greatest accuracy, it is generally desirable to use as few pulses as possible for patient convenience and comfort. A pulse is defined as any illumination of the retina, which may be a temporal illumination with any intensity, modulation and frequency.

Various pulse sequences may be utilized comprising, for example, a pulse or series of pulses at wavelengths of light that cause the breakdown (bleaching) of the visual pigment, and then a series of pulses (possibly with less intensity than the pulses that were used to cause the visual pigment breakdown) used to illuminate the retinal area of interest, allowing for the measurement of the change in reflection of the area of interest and, thus, the content of the visual pigment. This is shown in FIG. 12, with the bleaching pulse 46 and the smaller measurement pulses 41. The wavelength of the illuminating light could be the same as the initial bleaching light or the illuminating light could be of different wavelength than the bleaching light (requiring two separate light sources). One exemplary pulse sequence comprises one to four strong pulses, to heavily bleach the visual pigment, and then a series of low intensity pulses applied over a selected period of time to allow measurements to be made. The change in reflected light is measured via these measurements, and the change versus time indicates the rate of regeneration, as illustrated at 34 in FIG. 11. By measuring the slope of the regeneration curve, the glucose concentration can be calculated. The higher the slope of the regeneration curve of the visual pigment, the higher the concentration of glucose. This curve is not necessarily linear, because in some circumstances the measured rate of change of reflectance of the retina decreases as regeneration proceeds.

The wavelength of light chosen for the illumination pulses may be any wavelength that would be absorbed by any visual pigment. In a preferred method, narrow band light that is absorbed by either green, red, or blue visual pigment may be used. The device may use polychromatic light for the pulse sequence, with the light then being optionally filtered at the lenslet. Alternatively, narrow-band light specifically chosen for a particular visual pigment (e.g., 540 nm light for bleaching primarily of the green visual cone pigment) may be used as the illumination light. Narrow band light has the advantage that it is generally more comfortable for the patient.

When an area outside the fovea is interrogated by the measuring light, a background blue light (from a second source) may be used throughout the testing period to prevent the regeneration of the rod visual pigment, by keeping the rod pigment in a constant bleached state. Since the regeneration rate of this rod pigment is thought to be slower than cone visual pigment, combining the change in reflectance from the regeneration pigments with different regeneration rates may lessen the accuracy of the measurement without this feature.

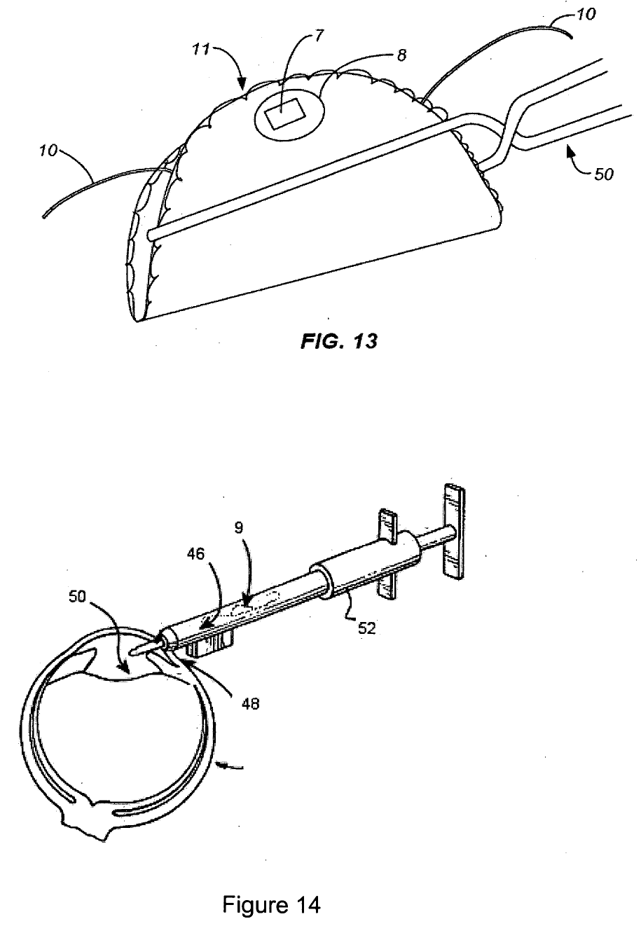

The IOL may be made of a rigid material such as polymethylmethacralate (PMMA). With a rigid IOL, the incision in the eye required is at least the diameter of the IOL, which in the range of approximately 6 mm to 10 mm. Recently, it has become desirable to have a smaller incision to reduce the complications from the surgical procedure. Foldable IOLs are now available. These allow the eye surgeon to make a smaller incision in the eye during the implantation procedure. FIG. 13 shows one example of a folded IOL 9, being held by a surgical instrument 50. One side of the IOL is shown, which in FIG. 13 reveals the LED 7 and the lenslet 8. The induction coil 11 is shown on the periphery of the IOL. The sensor is not shown in FIG. 13 and is in the folded portion of the IOL that is not in view. After implantation, the IOL is allowed to unfold and resume its natural shape inside the eye. Folded IOLs can be made of several different materials including acrylic and silicone. In this invention, the elements required for glucose measurement including the LED, sensor, lenslets, and the induction coils could be imbedded into the material for the folded IOL in a similar manner as they would be in the rigid IOL.

FIG. 14 shows one example of an inserter mechanism for insertion of an IOL into the posterior chamber 50 of the eye. The IOL 9 is folded into a compact shape and contained within the barrel 46 of inserter 52, which is then inserted into a small incision in the sclera 48 (the white colored section of the eye surrounding the iris), and the lens gently pushed into position. Over a period of seconds or minutes, the IOL relaxes to its original unfolded shape and remains in place.

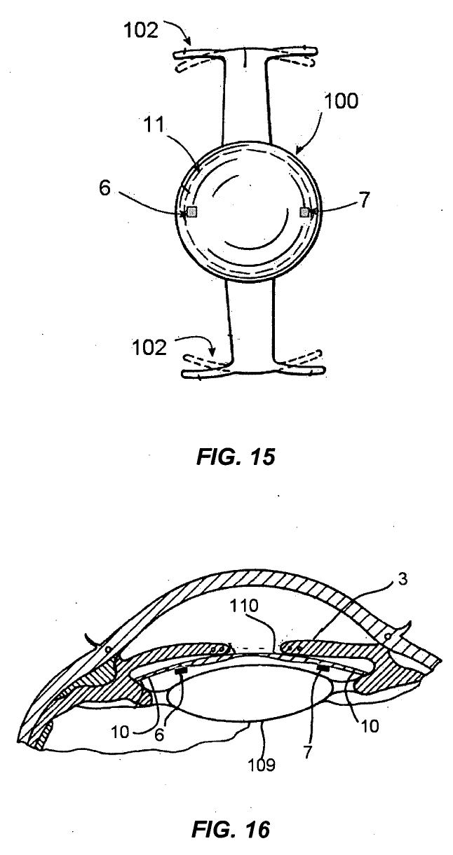

FIG. 15 illustrates an IOL designed to allow accommodation of the user by either changing in shape or position within the eye. IOL 100, which allows accommodation, contains specialized haptics 102 which help to secure the IOL within the eye, induction coil 11, LED source 7 and sensor 6.

FIG. 16 illustrates an intraocular contact lens containing elements of a glucose measurement system. Intraocular contact lens 110 is placed into the posterior chamber of the eye between iris 3 and natural lens 109. Haptics 10 hold the lens in place in the chamber, and LED 7 and sensor 6 serve as the source and detector for the glucose measurement.

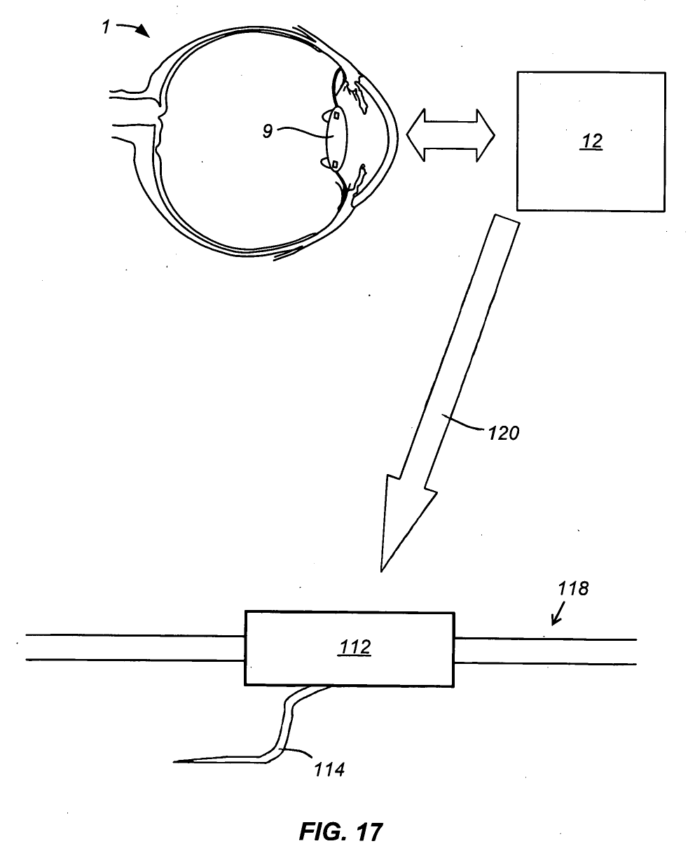

FIG. 17 shows an embodiment in which the blood glucose concentration monitor described above is used together with an insulin pump (or other insulin source) in a closed loop arrangement. The system has an IOL 9 implanted in eye 1 and interacting with an external fob 12 as described above. Blood glucose concentration information 120 is transmitted (such as via an IR link or other wireless link 120) to an insulin pump 112 residing, e.g., on a belt 118 worn by the subject. An appropriate amount of insulin based on the transmitted blood glucose concentration calculation is pumped into the subject through a cannula 114.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A blood glucose monitoring system comprising:
a light source adapted to transmit light onto at least a portion of a retina of an eye of a subject;
a sensor adapted to receive light from the retina;
a data capture and analysis system adapted to calculate blood glucose concentration of the subject from the light received by the sensor;
and support structure adapted to maintain positions of the light source and the sensor;
wherein at least one of the light source and the sensor is further adapted to be implanted within the eye.

2. The system of claim 1 wherein the light source is further adapted to transmit light in wavelengths absorbed by visual pigment.

3. The system of claim 1 wherein the light source is adapted to direct light onto a foveal region of the retina.

4. The system of claim 1 wherein the sensor is adapted to be implanted within the eye, the system further comprising a lens adapted to direct light from the retina onto the sensor.

5. The system of claim 1 wherein the sensor has a substantially toroidal shape and is adapted to be implanted within the eye.

6. The system of claim 1 wherein the sensor is adapted to be implanted within the eye and to transmit data outside of the eye to the data capture and analysis system.

7. The system of claim 1 wherein the light source and the sensor are both adapted to be implanted within the eye.

8. The system of claim 1 wherein the data capture and analysis system is adapted to be disposed outside of the eye.

9. The system of claim 8 wherein the data capture and analysis system further comprises a display adapted to display glucose concentration information.

10. The system of claim 8 wherein the data capture and analysis system further comprises a receiver adapted to receive information from the sensor.

11. The system of claim 10 wherein the receiver is further adapted to receive unique identification information from the sensor.

12. The system of claim 8 wherein the data capture and analysis system further comprises a transmitter adapted to transmit power to the light source.

13. The system of claim 12 wherein the data capture and analysis system transmitter is further adapted to transmit unique identification information to the sensor.

14. The system of claim 1 wherein the support structure comprises light source support structure adapted to support the light source behind an iris of the eye and in front of a natural lens of the eye.

15. The system of claim 1 wherein the support structure comprises light source support structure adapted to support the light source behind an iris of the eye and in place of a removed natural lens of the eye.

16. The system of claim 1 wherein the light source is adapted to be disposed outside the eye and the sensor is adapted to be implanted within the eye, the support structure comprising light source support structure adapted to support the light source in front of the eye and sensor support structure adapted to support the sensor within the eye.

17. The system of claim 1 wherein the support structure comprises light source support structure, the system further comprising an implant tool adapted to insert the light source and light source support structure into the eye.

18. The system of claim 1 further comprising an insulin source adapted to provide insulin to the subject in response to blood glucose concentration calculated by the data capture and analysis system.

19. The system of claim 18 wherein the insulin source comprises a pump.

20. The system of claim 1 further comprising an alarm adapted to provide an indication of an upcoming transmission from the light source.

21. The system of claim 20 further comprising a light transmission override adapted to be operated to temporarily prevent transmission from the light source to the subject.

22. The system of claim 1 further comprising an alarm adapted to provide notice to the subject as a result of a blood glucose concentration calculated by the data capture and analysis system.

23. A method of determining blood glucose concentration of a subject comprising:
transmitting light to a retina of an eye of the subject from a light source within the eye;
receiving reflected light from the retina; and
calculating blood glucose concentration from the reflected light.

24. The method of claim 23 further comprising activating an alarm based on a result of the calculating step.

25. The method of claim 23 wherein the transmitting step comprises directing light on a foveal portion of the retina.

26. The method of claim 23 wherein the receiving step comprises receiving light reflected from the retina in a sensor disposed within the eye.

27. The method of claim 23 wherein the receiving step comprises receiving light reflected from a foveal portion of the retina in a sensor disposed within the eye.

28. The method of claim 23 wherein the receiving step comprises receiving light reflected from the retina in a sensor disposed outside the eye.

29. The method of claim 23 wherein the calculating step comprises determining a rate of change of light reflected from the retina.

30. The method of claim 23 wherein the receiving step is performed by a sensor and the calculating step is performed at least in part by a data capture and analysis system, the method further comprising transmitting information related to light reflected from the retina from the sensor to the data capture and analysis system.

31. The method of claim 30 wherein the sensor is disposed within the eye and the data capture and analysis system is disposed outside of the eye, the step of transmitting information comprising transmitting the information from within the eye to outside the eye.

32. The method of claim 31 wherein the step of transmitting information comprises transmitting unique identification information from the sensor to the data capture and analysis system.

33. The method of claim 31 further comprising transmitting unique identification information from the data capture and analysis system to the sensor.

34. The method of claim 31 wherein the step of transmitting information comprises transmitting the information from within the eye to outside the eye through a closed eye.

35. The method of claim 23 further comprising transmitting power from a source external to the eye to the light source.

36. The method of claim 23 further comprising preventing ambient light from entering the eye during the receiving step.

37. The method of claim 23 further comprising displaying blood glucose concentration.

38. The method of claim 23 further comprising automatically controlling administration of insulin from an insulin source to the subject based on blood glucose concentration.

39. The method of claim 23 further comprising, prior to the transmitting step, providing notice to the subject of an upcoming transmitting step.

40. The method of claim 39 further comprising delaying the performance of the transmitting step.

41. A method of determining blood glucose concentration of a subject comprising:
transmitting light to a retina of an eye of the subject;
receiving reflected light from the retina in a sensor disposed within the eye; and
calculating blood glucose concentration from the reflected light.

42. The method of claim 41 further comprising activating an alarm based on a result of the calculating step.

43. The method of claim 41 wherein the transmitting step comprises directing light on a foveal portion of the retina.

44. The method of claim 41 wherein the transmitting step comprises transmitting light to the retina from a light source disposed outside the eye.

45. The method of claim 41 wherein the receiving step comprises receiving light reflected from a foveal portion of the retina.

46. The method of claim 41 wherein the calculating step comprises determining a rate of change of light reflected from the retina.

47. The method of claim 41 wherein the calculating step is performed at least in part by a data capture and analysis system external to the eye, the method further comprising transmitting information related to light reflected from the retina from the sensor to the data capture and analysis system.

48. The method of claim 47 wherein the step of transmitting information comprises transmitting the information from within the eye to outside the eye through a closed eye.

49. The method of claim 47 wherein the step of transmitting information comprises transmitting unique identification information from the sensor to the data capture and analysis system.

50. The method of claim 47 further comprising displaying blood glucose concentration.

51. The method of claim 41 wherein the calculating step is performed at least in part by a data capture and analysis system external to the eye, the method further comprising transmitting unique identification information from the data capture and analysis system to the sensor.

52. The method of claim 41 further comprising automatically controlling administration of insulin from an insulin source to the subject based on blood glucose concentration.

53. The method of claim 41 further comprising, prior to the transmitting step, providing notice to the subject of an upcoming transmitting step.

54. The method of claim 53 further comprising delaying the performance of the transmitting step.

55. A method of implanting a blood glucose monitor comprising:
providing an implantable blood glucose monitor comprising a light source;
inserting the implantable blood glucose monitor through an outside surface of an eye; and
orienting the light source in a position from which the light source can illuminate at least a portion of a retina of the eye.

56. The method of claim 55 wherein the providing step comprises providing an implantable blood glucose monitor comprising a light source and a sensor adapted to receive light, the orienting step further comprising orienting the sensor to receive light from the retina.

57. The method of claim 55 wherein the inserting step comprises supporting the implantable blood glucose monitor with an insertion tool.

58. The method of claim 57 wherein the supporting step comprises supporting the implantable blood glucose monitor in an insertion state, the method further comprising moving the implantable blood glucose monitor from the insertion state to a deployed state.

59. The method of claim 55 wherein the inserting step comprises inserting the implantable blood glucose monitor into an anterior chamber of the eye.

60. The method of claim 55 wherein the inserting step comprises inserting the implantable blood glucose monitor into a posterior chamber of the eye.

61. A blood glucose monitor comprising:
a receiver adapted to be disposed outside of an eye and to receive information related to light reflected from a retina of the eye from a sensor disposed within the eye; and
a processor adapted to calculate blood glucose concentration from the information.

62. The blood glucose monitor of claim 61 wherein the receiver is further adapted to receive information related to light reflected from the sensor through a closed eye.

63. The blood glucose monitor of claim 61 further comprising a display adapted to display blood glucose concentration calculated by the processor.

64. The blood glucose monitor of claim 61 further comprising a transmitter adapted to transmit power to a light source implanted in the eye.

65. The blood glucose monitor of claim 61 further comprising a transmitter adapted to transmit a signal related to calculated blood glucose concentration to an insulin source.

66. A blood glucose monitor comprising:
a sensor adapted to be disposed outside of an eye and to receive light reflected from a retina of the eye from a light source disposed within the eye; and
a processor adapted to calculate blood glucose concentration from the information.

67. The blood glucose monitor of claim 66 further comprising a display adapted to display blood glucose concentration calculated by the processor.

68. The blood glucose monitor of claim 66 further comprising a transmitter adapted to transmit power to a light source implanted in the eye.
